(19) United States
(12) Patent Application Publication
(10) Pub. No.: US 2003/0236450 A1
(43) Pub. Date: Dec. 25, 2003

(54) System for remotely monitoring and controlling CPAP apparatus

(76) Inventor: Richard J. Kocinski, North Huntingdon, PA (US)

Correspondence Address: MACMILLAN SOBANSKI & TODD, LLC, ONE MARITIME PLAZA FOURTH FLOOR, 720 WATER STREET, TOLEDO, OH, 43604-1619, US

(21) Appl. No.: 10/393,169

(22) Filed: Mar. 20, 2003

Related U.S. Application Data
(60) Provisional application No. 60/390,936, filed on Jun. 24, 2002.

(51) Int. Cl. G06F 17/60; A61B 5/00

(52) U.S. Cl. 600/300; 705/2

(57) ABSTRACT

A data collection and processing system provides user and apparatus information from a CPAP apparatus to a remote location. The system also allows the operating settings for the CPAP apparatus to be remotely programmed. Information is collected and stored in a digital memory in the CPAP apparatus. The CPAP apparatus is programmed to periodically transfer data to a central database server. The central database server can collect and store data from CPAP apparatus for a number of patients who undergo sleep therapy. A user can access the central database server over the internet to obtain information. Each user is given access only to information for his or her patients. The user may provide data to the central database server for transmission to the CPAP apparatus during a subsequent connection that the CPAP apparatus makes to the central database server.

### CROSS REFERENCE TO RELATED APPLICATIONS

This application claims the benefit of U.S. Provisional Patent Application No. 06/390,936 filed Jun. 24, 2002.

### BACKGROUND OF THE INVENTION

Sleep apnea is generally caused by a blockage, which impedes inhalation, of a person's airway during sleep. A sleeping person having an apnea event will struggle and may partially awaken until a breath can be taken. In people with severe sleep apnea, there can be a significant reduction in blood oxygen saturation, which can cause health problems, such as heart disease. A CPAP apparatus is used to apply a continuous positive airway pressure to a person during sleep to treat sleep-related illnesses, such as sleep apnea. The apparatus includes a turbine connected through a hose to a nasal mask to apply a positive pressure to the patient's airway. The positive pressure is applied at least during inhalation and, frequently, during both inhalation and exhalation. The positive airway pressure tends to sufficiently inflate the patient's airway to prevent blockages.

Many patients do not recognize that they are suffering from sleep apnea. Unless someone else recognizes and tells them that they have a problem, they may only be aware of the fact that they are always tired and that they do not sleep well. An accurate diagnosis and prescription for treatment for sleep disorders is generally determined by having the patient spend one or more nights at a sleep clinic where the patient is observed by a medical professional during sleep. Once the problem is diagnosed, the patient may be connected to a CPAP apparatus that is used to deliver an optimum positive pressure setting for preventing apnea events.

Many patients initially have trouble adjusting to sleep while connected through a mask to the CPAP apparatus. As a consequence, patient compliance with newly prescribed CPAP therapy may be low. A medical professional needs to know if the patient is using the CPAP apparatus at night as prescribed and if the pressure level is proper for preventing apnea events. It is desirable that the pressure be set to the lowest effective pressure, since patient discomfort increases with increased pressure.

There are three types of compliance monitoring currently in use. The first type is subjective monitoring, wherein the monitoring is based on the patient's reports, which are compiled by a visit with the patient or a telephone call. Another type is objective monitoring, which is based on time treated with a CPAP apparatus. This type of monitoring is typically based on readouts from an onboard CPAP apparatus hour meter. The last type is effective monitoring, which is based on the actual time the patient is treated with an appropriate pressure. This is the most effective type of monitoring. However, it has inherent difficulties, such as the added expense for special CPAP monitoring units, complicated procedure and overhead costs for collecting data, and patient involvement, as the patient must be relied upon to return the special CPAP monitoring unit.

What is needed is an easy, efficient, effective, and economical compliance monitoring system.

It would be desirable if a compliance monitoring system made available compliance information to the medical professional's office so that data collection could be made with fewer patient visits. Such a system would further minimize attention on compliant patients, rendering more time to spend on non-compliant patients.

It also would be desirable for the medical professional to set the CPAP output pressures and other operating parameters from a remote location. Since the CPAP apparatus may be leased or may need periodic service, a further desired feature would be to have service information available to the medical equipment provider from a remote location.

### BRIEF SUMMARY OF THE INVENTION

The invention is directed to a data collection and processing system that provides user and apparatus information from a CPAP apparatus in a patient's home to a medical professional and to a medical equipment provider at remote locations. The system also allows the medical professional to remotely program the operating settings for the CPAP apparatus. Information is collected and stored in a digital memory in the CPAP apparatus as the patient uses the CPAP apparatus. The CPAP apparatus is programmed to periodically transfer data via a modem and a telephone connection to a central database server. The central database server can collect and store data from CPAP apparatus for a number of patients who undergo sleep therapy.

The medical professional can access the database server over the internet to obtain information on patient compliance and, preferably, other information, such as a patient's response to CPAP treatments. Each medical professional is given access only to information for his or her patients. Preferably, the medical professional also may provide data to the central database server for transmission to the CPAP apparatus during a subsequent connection that the CPAP apparatus makes to the central database server. This information may include, for example, the frequency at which the CPAP apparatus connects to the central database server, and the level of pressures applied to the patient. The central database server also may be accessed over the internet by the medical equipment provider for checking service information, and by a reimbursement organization for verifying payments that are due to the medical equipment provider and/or the medical professional. Further, the central database system can be programmed to automatically send email notices to the medical equipment provider when the CPAP apparatus requires servicing and to inform the medical professional when updated data has been received from the CPAP apparatus for one of his or her patients.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a diagrammatic view of a system according to a preferred embodiment of the invention for providing information relating to usage of a CPAP apparatus in a patient's home to remote locations and to allow remote programming of operational settings for the CPAP apparatus.

FIG. 2 is a flowchart depicting a method for setting up a central database server according to a preferred embodiment of the invention.

FIG. 3 is an image of a “Modem Inventory” window of a windows-based, software-driven internet processing system for use in setting up the central database server.

FIG. 4 is an image of a “Device Inventory” window of the internet processing system.

FIG. 5 is an image of a “Login” window of the internet processing system.

FIG. 6 is an image of a “Subscription” window of the internet processing system.

FIG. 7 is an image of a “Patient” window of the internet processing system wherein new patient information may be entered.

FIG. 8 is an image of the “Patient” window of the internet processing system wherein a device/modem may be assigned to a patient.

FIG. 9 is a front perspective view of a modem and CPAP apparatus according to the present invention connected in a patient's home.

FIG. 10 is an image of the “Patient” window of the internet processing system and a report accessible by authorized users by clicking a “Report” button in the “Patient” window.

FIG. 11 is an image of the “Patient” window of the internet processing system and a “Device Settings” wherein an authorized user can enter and change the settings of a CPAP device.

FIG. 12 is an image of a “Direct Connection” wherein the internet processing system can be used without a modem.

### DETAILED DESCRIPTION OF THE INVENTION

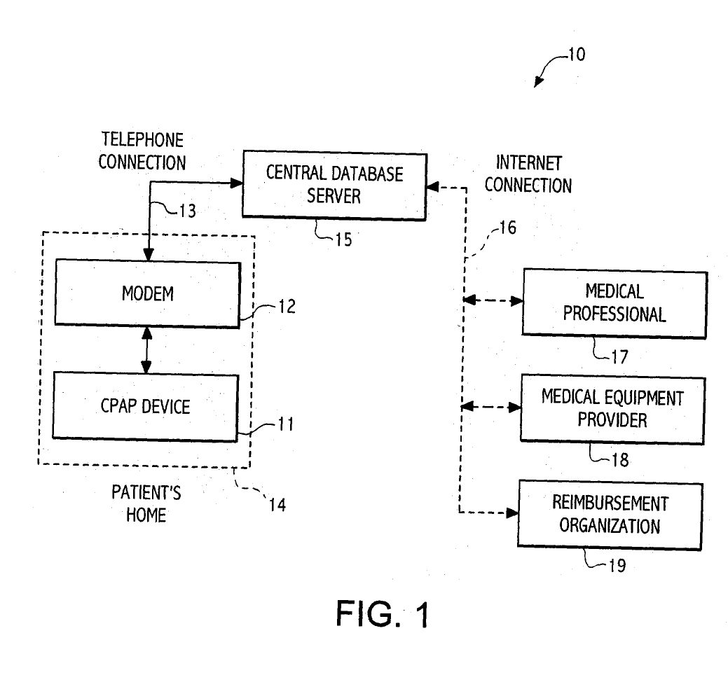

Now with reference to the drawings, there is illustrated in FIG. 1 a system for providing communications between a CPAP apparatus located in a patient's home or premises and a medical professional, and/or a medical equipment provider and, optionally, a medical insurance or other reimbursing organization. A diagram of a system 10 for remotely monitoring and controlling the CPAP apparatus 11 is shown in the attached drawing. The CPAP apparatus 11 is provided with a modem 12 for use in connecting the CPAP apparatus 11 to a telephone line 13. The CPAP apparatus 11 and modem 12 are adapted to be located in a patient's home 14. The modem 12 can be programmed at set times to dial a telephone number for a central database server 15 and to transfer information to and from the central database server 15. It will be appreciated that a large number of widely dispersed CPAP apparatus 11 may be connected to dial the central database server 15, and that the central database server 15 may be connected to one or to a number of telephone lines for receiving data from the CPAP apparatus 11. For example, a single central database server 15 may be used for collecting and storing information for a large number of CPAP apparatus 11 located in a multi-state region or in one or more countries.

Operating information and programming information may be stored in a memory either in the CPAP apparatus 11 or in the modem 12. The modem 12 also may be provided with a pass through operating mode, which permits the transfer of real time information from and to the CPAP apparatus 11.

Data transfer between the CPAP apparatus 11 in the patient's home 14 and the central database server 15 is preferably via telephone lines 13 due to their availability in homes. It should be appreciated that the data transfer also may be made through other available communications channels, for example, via a cable modem and the internet or via a satellite communications system where such service is available and cost effective.

The central database server 15 also can be connected to the internet, which is represented by a dashed line 16. A medical professional 17, a medical equipment provider 18 and, optionally, a medical reimbursement organization 19 may use the internet to access information in the central database server 15 via a communications network, preferably via the internet 16. A record is set up in the central database server 15 for each CPAP apparatus 11. The record can include, for example, information identifying the patient, information identifying the model and serial number and location of the CPAP apparatus 11, information identifying the medical professional 17 responsible for the patient, information identifying the medical equipment provider 18, information identifying any reimbursement organization 19, information downloaded from the CPAP apparatus 11, and programmable settings for the CPAP apparatus 11. The central database server 15 can be programmed to provide selective access to a patient's record so that each party authorized to access the record is provided with access only to information that party needs. If the record includes information identifying the patient, it may be encoded to protect the patient's privacy.

Depending on the sophistication of the CPAP apparatus 11, different amounts of data may be collected and transferred to the central database server 15. At a minimum, it is preferable that the CPAP apparatus 11 collect and transfer information on patient usage compliance of the CPAP apparatus 11. Information may be collected and stored regarding the output pressure, startup delays and pressures in which a lower pressure is initially applied to the patient, and the frequency and nature of events detected while the patient sleeps, such as apnea events, maintenance warnings. The CPAP apparatus 11 may be programmed, for example, to send a message to the central database server 15 which is relayed to the medical professional 17 if the number of apnea events per hour exceed a programmed threshold, or if the number of hypoapnea events per hour exceed a programmed threshold, or if the blood desaturation exceeds a programmed threshold. The CPAP apparatus 11 also can be programmed to send a message to the medical professional 17 via the central database server 15 if there is no or very little patient usage of the CPAP apparatus 11 over a predetermined period of time, such as a preset number of days. The medical professional 17 may program multiple thresholds for the patient in the CPAP apparatus 11. The maintenance warnings may be made available only to the medical equipment provider 18 and the pressures and sleep events may be made available only to the medical professional 17, since others do not need this information. The CPAP apparatus 11 may be programmed to automatically send a warning to the central database server 15 anytime an operating problem is detected. For example, if the CPAP apparatus 11 ceases operation or if it cannot provide an adequate pressure, a warning message can be sent.

When the modem 12 and transfers data to the central database server 15, the server 15 can be programmed to send messages to the appropriate person or organization via the internet. For example, the medical professional 17 will receive an automatic email message indicating that there is new compliance data stored in the central database server 15. Anytime an operational or maintenance warning is received, a message reporting the warning can be automatically forwarded via email to the medical equipment provider 18.

The medical professional 17 also can program the operation of the CPAP apparatus 11 via the internet. For example, if it is noted by the medical professional 17 that too many abnormal sleep events are occurring during the patient's sleep, the programmed operating pressure setting may need to be increased. This information is sent by the medical professional 17 via the internet to the central database server 15 and stored with the patient/equipment record. The next time the CPAP apparatus 11 calls the server, the new pressure setting will be downloaded into the apparatus 11 to program the new pressure level. The medical professional 17 also may program the frequency at which the CPAP apparatus 11 calls the central database server 15. When CPAP therapy is first prescribed, the medical professional 17 (e.g., a physician or referral lab) may want to receive a daily report to verify that the patient is using the equipment and that the settings are correct for the patient. This can be done without generating paperwork. After a week or two, the call back or reporting interval may be lengthened. For a patient who has been successfully using the apparatus 11 for some time, an even longer reporting interval may be set.

The CPAP apparatus 11 is programmed to transfer data to the central database server 15 at intervals set by the medical professional 17. The CPAP apparatus 11 also may be programmed to call the central database server 15 at times other than the programmed times. For example, a call may be made if there is a service problem or if the patient is experiencing too many abnormal sleep events. If a call is triggered by events in a patient's condition that may require reprogramming the CPAP apparatus 11, the CPAP apparatus 11 may go into a mode where it calls the central database server 15 more frequently in order to more timely receive any new programming information from the medical professional 17.

The central database server 15 may be programmed to use any one or more of several different procedures for initially setting up a record in the central database server 15 for new CPAP apparatus 11. According to a first procedure, the medical equipment provider 18 will deliver and install the CPAP apparatus 11 in the patient's home 14 and will program the CPAP apparatus 11 according to the medical professional's 17 prescription. After delivery and set up is completed, the CPAP apparatus 11 will be activated to make an initial call to the central database server 15. A record will be set up with information stored in the CPAP apparatus 11, such as its settings, the identification of the medical equipment, and the identity of the medical professional 17 given access to the record.

According to a second procedure, the medical equipment provider 18 may use the internet to set up a record in the central database server 15 prior to installation of the CPAP apparatus 11. The information may include, for example, information identifying the CPAP apparatus 11, programming settings prescribed by the medical professional 17 and the identity of those given access to the record. After the CPAP apparatus 11 is delivered and set up, a technician causes the CPAP apparatus 11 to dial the central database server 15 and programming information is downloaded to the CPAP apparatus 11.

According to a third procedure, the medical professional 17 may use the internet to set up a record in the central database server 15 with information including the patient's name and home address, the type of CPAP apparatus 11 needed, the programming settings for the CPAP apparatus 11, and the identity of the medical equipment provider 18. The central database server 15 may then send an email placing an order with the designated medical equipment provider 18. After the CPAP apparatus 11 is delivered and set up, it is caused to call the central database server 15 to download the programming settings for the patient. If there is a reimbursement organization, the central database server 15 also may be programmed to automatically send a message identifying the patient and confirming that the CPAP apparatus 11 has been delivered and installed in response to the downloading of the initial programming information.

Two-way communication between the CPAP apparatus 11 and the central database server 15 is accomplished via an internet processing system (IPS). The IPS collects compliance data and other parametric data from the CPAP apparatus 11 in the patient's home 14, transmits the data to a central database server 15, for example, by the telephone connection 13 via the modem 12, and makes the data available to the medical professional 17, the medical equipment provider 18, and the reimbursement organization 19 via the internet connection 16. The IPS permits the medical professional 17 and the equipment provider 18 to control the parameters of the CPAP apparatus 11 via the internet connection 16 to the central database server 15 and further to the CPAP apparatus 11 in the patient's home 14 via the telephone connection 13. Additionally, the IPS prompts the central data server 15 to generate email notifications, which confirm actions, alert of specific conditions within user defined thresholds, and notify of information postings, to the medical professional 17 and the equipment provider 18.

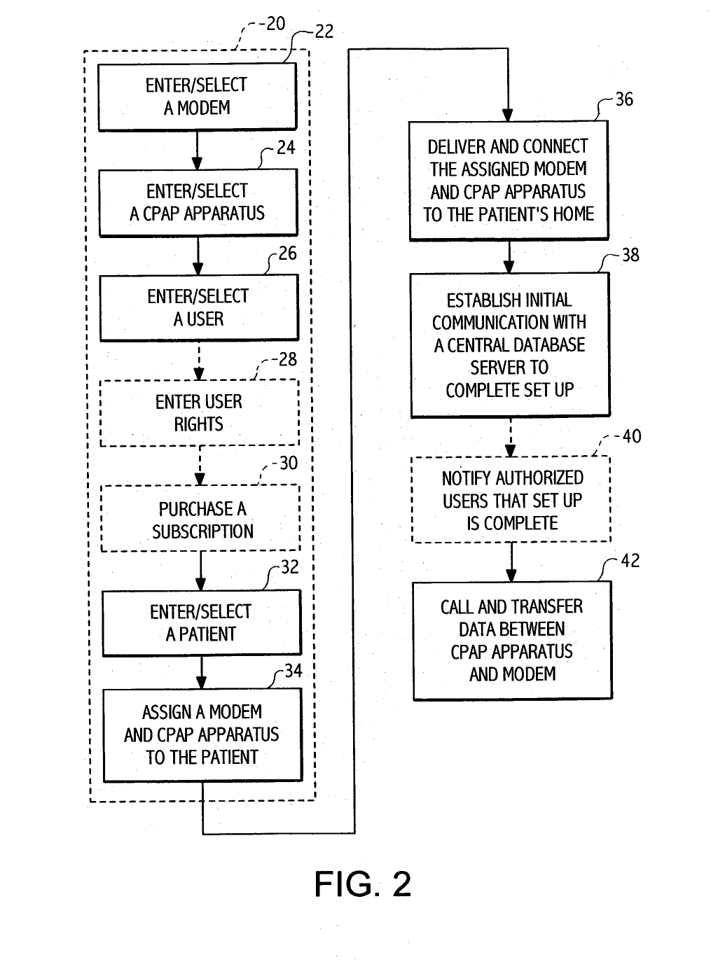

The central database server 15 can be set up, as indicated at function step 20 in FIG. 2, via the IPS by entering the modem 12 and the CPAP apparatus 11 into inventory, entering the authorized users and viewers, purchasing a subscription service, entering the patient information, and assigning the CPAP apparatus 11 and modem 12 to a patient. The system 10 set up is completed by delivering and connecting the CPAP apparatus 11 and modem 12 to the patient's home 14.

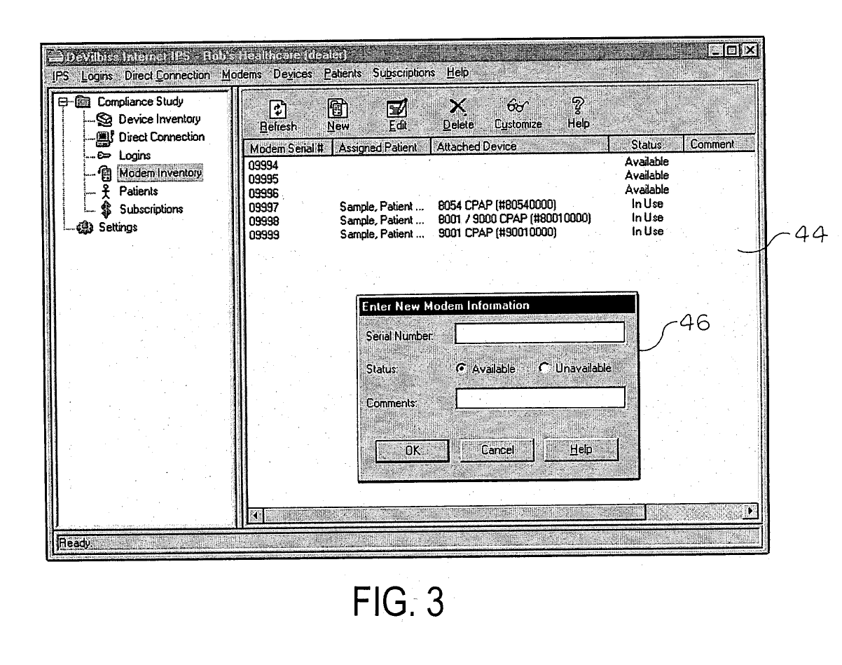

The IPS can be a software-driven system. The software can be in the form of a windows-based application having point-and-click menu options that can easily be selected by the user. The software can be launched by clicking an icon on the desktop. New modems can be entered, as indicated in function step 22, and modems available and in use can be displayed in a “Modem Inventory” window 44, as shown in FIG. 3. The “Modem Inventory” window 44 can be opened by selecting “Modem Inventory” from a menu selection. To add a new modem, simply click the “New” button in the “Modem Inventory” window 44. An “Enter New Modem Information” window 46 will solicit new modem information, such as the modem serial number, status, and optional comments. After entering the solicited information, click the “OK” button to save the information and close the window or the “Cancel” button to close the window without saving the information. Modems entered will show up on the list in this window. Patient links will be shown up in this window after those links are made.

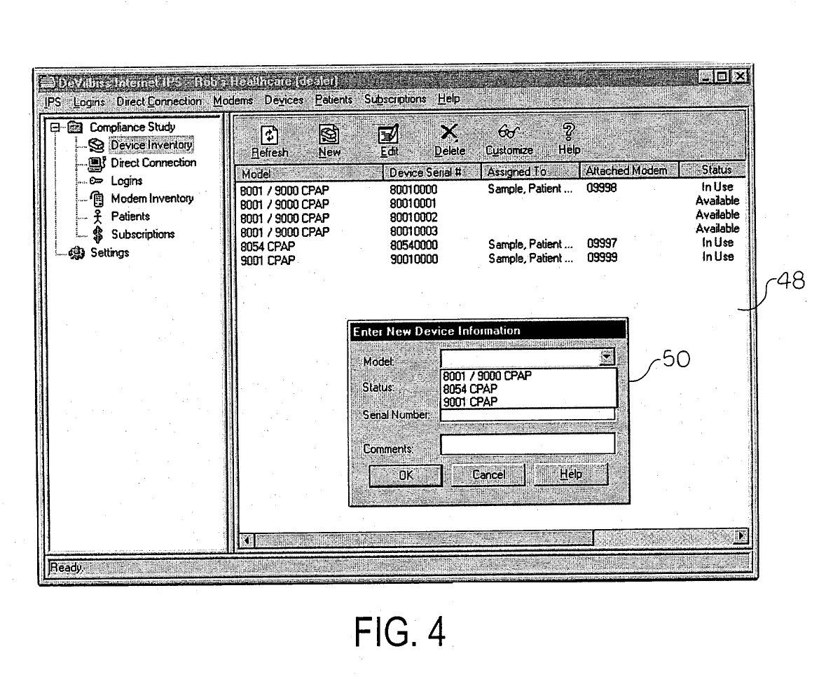

Similarly, a “Device Inventory” window can be opened to enter new CPAP apparatus 11, as indicated in function step 24, and display CPAP apparatus 11 available and in use. The “Device Inventory” window 48, as illustrated in FIG. 4, can be opened by selecting “Device Inventory” from a menu selection. To add a new CPAP apparatus 11, click the “New” button. An “Enter New Device Information” window 50 will solicit new CPAP apparatus 11 information, such as the model, status, serial number, and optional comments. After entering the solicited information, click the “OK” button to save the information and close the window or the “Cancel” button to close the window without saving the information. CPAP apparatus 11 entered will show up on the list in this window. Patient links will be shown up in this window after those links are made.

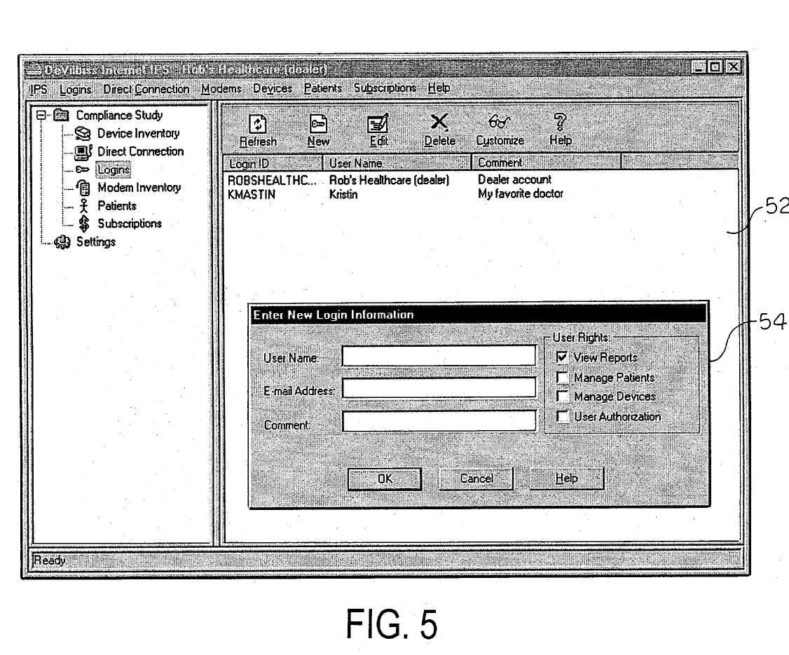

Authorized users and viewers can be entered, as indicated in function step 26, through a “Login” window 52, as shown in FIG. 5, that can be opened by selecting “Login” from a menu selection. To make a new entry, click the “New” button in the “Login” window. An “Enter New Login Information” window 54 will solicit new login information, such as the user name, email address, and optional comments. User rights, such as permission to view reports, manage patients, manage devices, and user authorization, can be entered, as indicated in function step 28, for example, by selecting corresponding boxes in the “New Login Information” window. A user who is authorized to view reports has read only access. Such a user can only view and print patient, device, modem, and compliance information. A user who is authorized to manage patients can add, edit, or remove patient information, assign CPAP apparatus 11 and modems 12 to patients, change CPAP apparatus 11 settings, and order patient subscriptions. A user who is authorized to manage devices can add and remove CPAP apparatus 11 and modems 12. A user with user authorization can set up other authorized users who can access the system. After entering the user information, click the “OK” button to save the information and close the window or the “Cancel” button to close the window without saving the information. Users entered will show up on the list in this window. Linking to patients will occur during patient set up. Existing user rights can be opened and edited as desired.

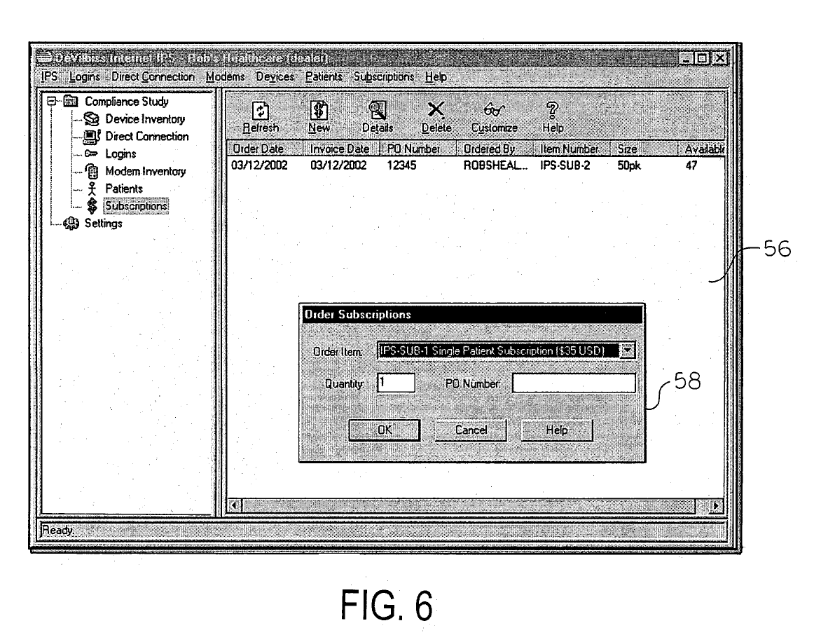

Subscriptions to use the system 10 can be purchased, as indicated in function step 30, through a “Subscription” window 56, as shown in FIG. 6, that can be opened by selecting “Subscription” from a menu selection. To purchase subscriptions, click the “New” button in the “Subscription” window. An “Order Subscriptions” window 58 will solicit information, such as item, quantity, and purchase order number. Click the “OK” button to save the order subscription and close the window or the “Cancel” button to close the window without saving the order. Subscriptions used and available will show up on this list.

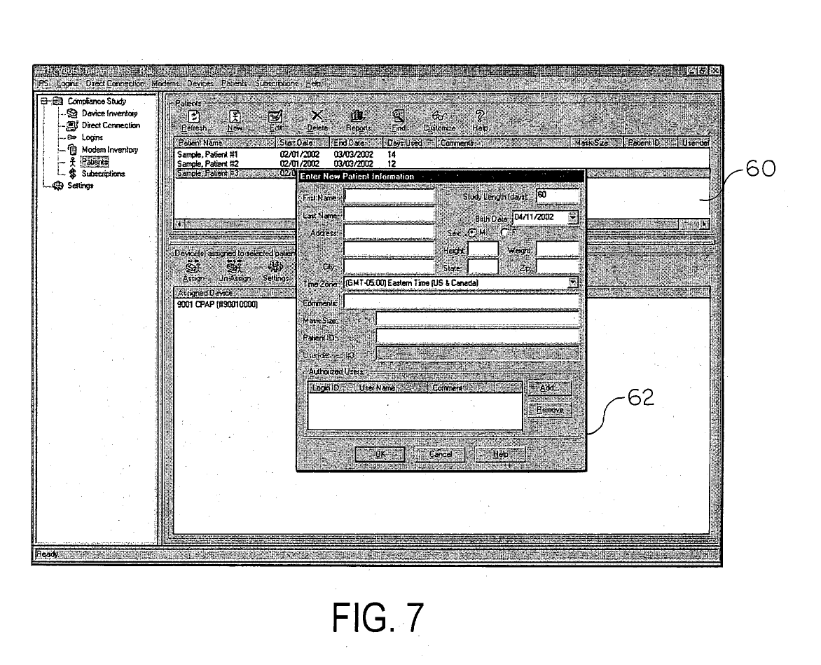

A new patient can be entered or selected, as indicated at function step 32, and patient information can be accessed through a “Patient” window 60, as shown in FIG. 7, that can be opened by selecting “Patient” from a menu selection. Patients already entered will be displayed in a list and can be edited by highlighting the patient and clicking the “Edit” button. A patient search can be performed by clicking the “Find” button. New patients can be added by clicking the “New” button in the “Patient” window. An “Enter New Patient Information” window 62 will solicit information, such as name, address, date of birth, gender, height, weight, time zone, comments, mask size, patient identification, study length in days, and authorized users. Authorized users can be added or removed by clicking “Add” or “Remove” buttons. Clicking the “Add” button produces a window containing a list of authorized viewers. The level of authorization is created by entering authorized users and viewers, as set forth above. Click the “OK” button to save the patient entry and close the window or the “Cancel” button to close the window without saving the entry. User defined fields can be set up by clicking on the “Settings” button (i.e., in the left column when viewing the drawing) and the “Global Dealer Preferences” (not shown).

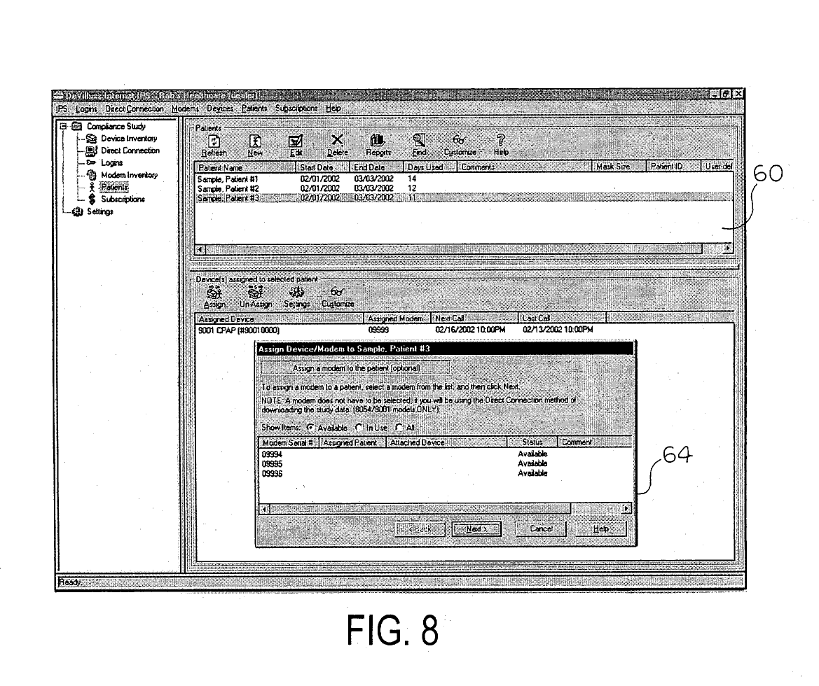

CPAP apparatus 11 and modems 12 are assigned to patients, as indicated in function step 34, in the “Patient” window by highlighting the patient being assigned and clicking on the “Assign” button. This opens an “Assign Device/Modem” window, as shown in FIG. 8. Check the “Available” filter, select a modem 12 from the list and click the “Next” button. Check the “Available” filter, select a CPAP apparatus 11 from the list and click the “Next” button. Enter initial device setting values in “Settings”. When the CPAP apparatus 11 and the modem 12 are prompted to make an initial call to the central database server 15, these initial settings may be communicated to the CPAP apparatus 11. When the patient is highlighted in the upper window, the assigned inventory will be shown in the lower window. At the end of the compliance period, once again highlight the patient and assigned device/modem and then click “Un-Assign” to free up the inventory for another patient.

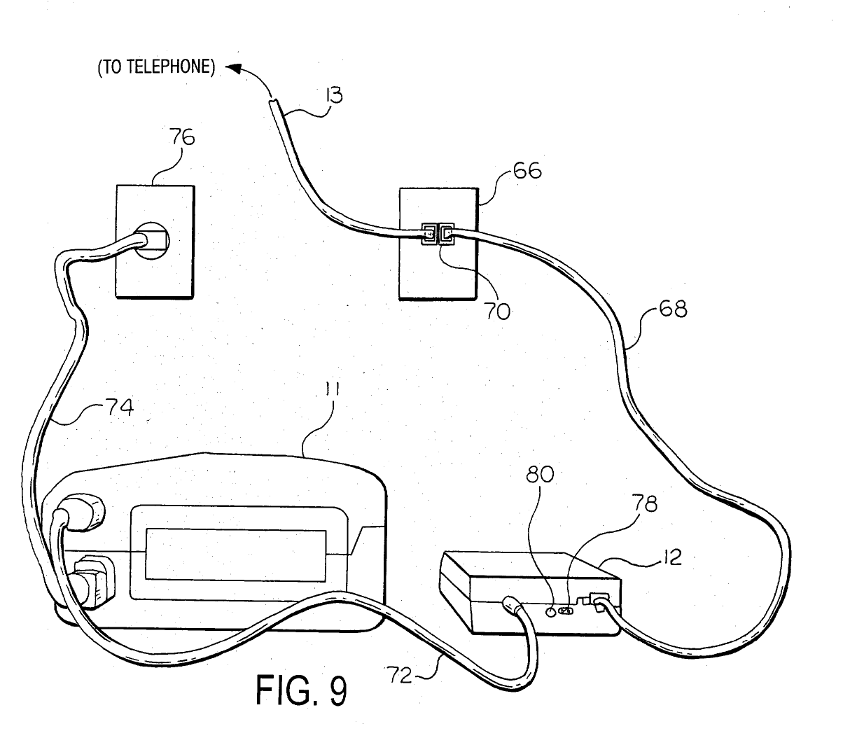

At this juncture, the patient is set up on the central database server 15 and the CPAP apparatus 11 and modem 12 can be delivered and connected to the patient's home 14, as indicated in function step 36. The modem 12 is connected to a standard household telephone jack 66 by a telephone line 68 as shown in FIG. 9. The modem 12 can share the telephone jack 66 with a telephone (not shown) by removing the telephone line 13 from the telephone jack 66 and placing a splitter adapter 70 in the telephone jack 66, as shown in FIG. 9. The telephone line 13 can be plugged into one side of the splitter adapter 70 and the modem telephone line 68 can be plugged into the other side of the splitter adapter 70. The modem 12 is connected to the CPAP apparatus 11 with the modem cable 72 and the power cord 74 from the CPAP apparatus 11 is connected to a power outlet 76.

After the CPAP apparatus 11 and modem 12 are connected to the patient's home 14, the modem 12 is prompted to make an initial call to the central database server 15, as indicated in function step 38 in FIG. 2. This can be accomplished by simply pressing a “Call” button 78 on the modem 12. Visual indicators can provide an indication of the operation of the modem 12. For example, upon pressing the “Call” button 78, a light emitting diode (LED) 80 will light up. Upon releasing the “Call” button 78, the LED 80 will flash every few seconds, indicating that a call is in process. If the call is successful, the LED 80 will light continuously. The LED 80 will stay lit for a period of time (e.g., one minute) after the call is completed. The medical equipment provider 18 will receive an “assignment complete” message, as indicated in function step 40. The authorized users and viewers will receive an email notification when usage information is communicated from the CPAP apparatus 11 to the central database server 15. The medical equipment provider 18 can establish a delay period, in which the medical equipment provider 18 can work with the patient before sending the notification out.

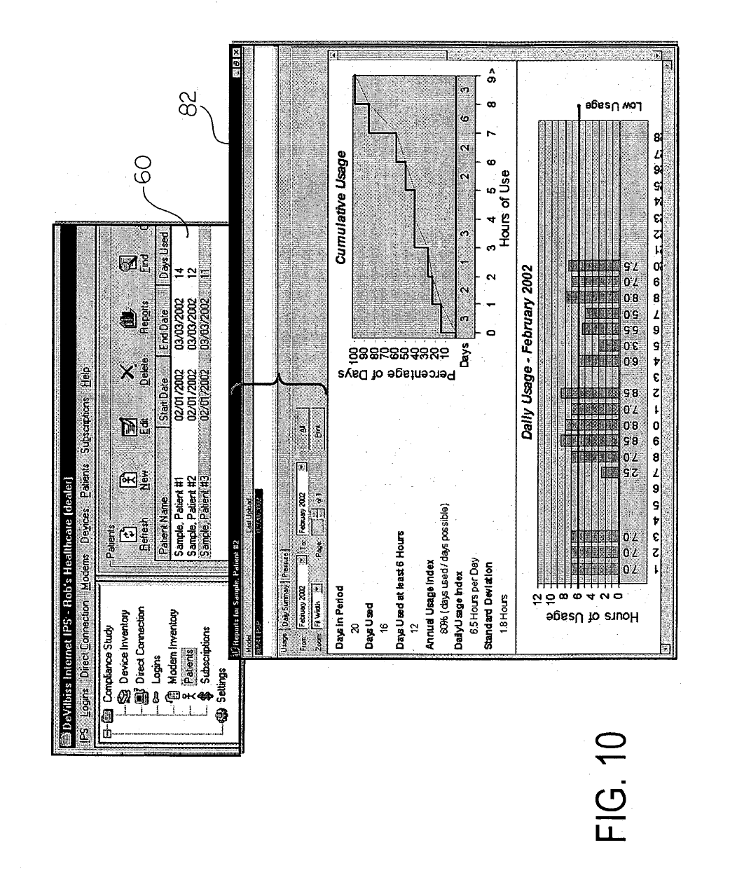

After the initial call to the central database server 15, the modem 12 can automatically call in data to the center database server 15 at certain time intervals, as indicated in function step 42. During the call, data will be immediately transferred from the CPAP apparatus 11 to the central database server 15, which stores and analyzes usage compliance data. If non-compliance behavior is detected, the medical professional 17 can be remotely notified by email. Authorized users will get reports, such as the report 82 as illustrated in FIG. 10. Reports can be viewed by selecting a patient from the “Patient” window 60. This is accomplished by highlighting the patient and then clicking the “Reports” button. To view the patient's compliance data, the viewer can scroll through and select a period, such as a month, or select all and print a graph. Reports will vary depending on the model of the CPAP apparatus 11. The patient's name will appear in the title bar of the window. Tabs can be used to move between reports.

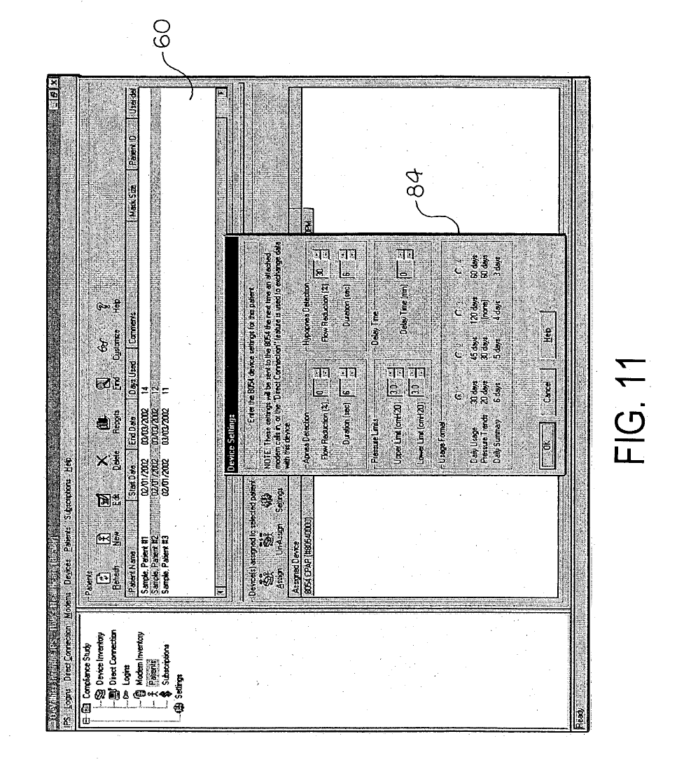

According to a preferred embodiment of the invention, the CPAP apparatus 11 can be controlled remotely. This is accomplished by highlighting a patient and then highlighting the assigned devices and then clicking the “Settings” button. A “Device Settings” window 84, as indicated in FIG. 11, will open. An authorized user can make changes as desired. The next time the modem 12 calls, the CPAP apparatus 11 will be updated. This feature may be available only on certain model CPAP apparatus 11, such as a CPAP apparatus 11 that has auto-adjust capabilities, which uses an internal pressure transducer and has internal memory that allows a range of pressure to be used and will adjust to give the patient more or less pressure, depending on their needs through the night. Such a CPAP apparatus 11 will react to the patient's sleep and breathing patterns for maximum comfort.

The patient is linked to the serial numbers on the CPAP apparatus 11 and modem 12. Consequently, the serial numbers that are entered during patient set up must match the serial numbers on the CPAP apparatus 11 and modem 12 that is delivered to the patient's home 14. The central database server 15 is secure with a firewall and access to patient information is password and login-protected to provide security for the patient's information. The user can customize the password and the screen view according to individual preference, desired information, and use.

The system 10 according to the present invention combines modem technology with the power of the internet to deliver fast, timely, and accurate data on the patient's use of the CPAP apparatus 11. By receiving timely non-compliance notification, early patient intervention is possible. This allows the medical professional 17 to spend time managing non-compliance instead of trying to identify it. Timely non-compliance notification also increases the likelihood of long-term compliance.

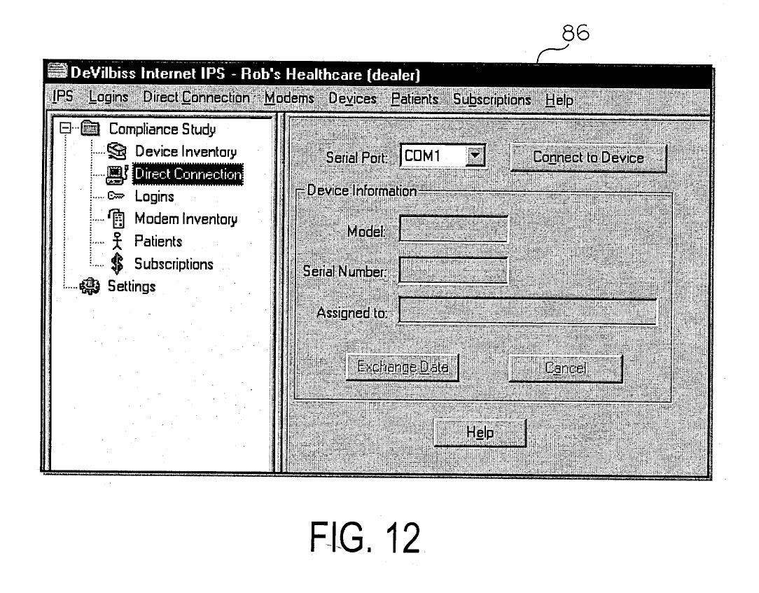

It should be appreciated that the CPAP apparatus 11 can optionally be connected directly to a computer (not shown). The computer can communicate with the central database server 15 via the modem 12 or can have the same software as the central database server 15 and thus function as the central database server 15 and bypass the modem 12. The direct connection can be set up via a “Direct Connection” window 86, as shown in FIG. 12, by selecting “Direct Connection” from the menu selection of the IPS. This will establish communication between the CPAP apparatus 11 and the computer. Compliance data can be downloaded to the computer and settings can be sent to the CPAP apparatus 11 via an internet connection 16 simply by pressing the “Exchange Data” button in the “Direct Connection” window.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A system for providing communications between a CPAP apparatus and a central database server at a remote location, the system comprising:
a central database server;
a CPAP apparatus; and
a modem connecting the CPAP apparatus to a telephone line for providing communication between the CPAP apparatus and the central database server, the system being programmed to dial a telephone number for a central database server and to transfer information between the CPAP apparatus and the central database server.

2. A system according to claim 1 wherein the CPAP apparatus and modem are located in a patient's home.

3. A system according to claim 1 wherein the communication remotely monitors and controls CPAP apparatus.

4. A system according to claim 1 wherein the central database server collects and stores information from a plurality of other widely dispersed CPAP apparatus connected to a number of other telephone lines.

5. A system according to claim 1 wherein at least one of either the CPAP apparatus or the modem has memory for storing operation and programming information.

6. A system according to claim 1 wherein the CPAP apparatus collects information on a patient's usage compliance of the CPAP apparatus and transfers the information to the central database server.

7. A system according to claim 1 wherein the central database server is programmed to relay a message to a location remote from the central database server and the CPAP apparatus.

8. A system according to claim 1 wherein operation of the CPAP apparatus can be remotely programmed via an internet connection through the modem.

9. A system comprising:
a central database server;
a CPAP apparatus;
a modem connecting the CPAP apparatus to the central database server in a remote location for providing communication between the CPAP apparatus and a central database server; and
a computer located remotely from the CPAP apparatus and the central database server, the computer being adapted to be connected to the central database computer via an internet connection.

10. A system according to claim 9 wherein the modem is a cable modem for connecting the CPAP apparatus to one of a cable or satellite internet connection.

11. A system according to claim 9 wherein the CPAP apparatus is one of a plurality of CPAP apparatus each having a record on the central database server.

12. A system according to claim 9 wherein the central database server is programmed to provide selective access to a patient's record.

13. A system according to claim 9 wherein the CPAP apparatus collects information on a patient's usage compliance of the CPAP apparatus and transfers the information to the central database server.

14. A system according to claim 9 wherein at least one of either the CPAP apparatus or the modem is programmed to send a message to the central database server where the message is relayed to the computer.

15. A system according to claim 9 wherein the operation of the CPAP apparatus can be programmed via an internet connection.

16. A system comprising:
a CPAP apparatus; and
a computer connected to the CPAP apparatus, the computer having a memory for storing operation and programming information for the CPAP apparatus, the CPAP apparatus being adapted to collect information on a patient's usage compliance of the CPAP apparatus and transfers the information to the computer.

17. A system according to claim 16 further including a modem for connecting the computer to a central database server, the computer being programmed to send a message to the central database server where the message is relayed to another remote location.

18. A system according to claim 16 wherein operation of the CPAP apparatus can be remotely programmed via an internet connection through a modem.

19. A method for remotely monitoring and controlling CPAP apparatus, comprising the steps of:
a) setting up a central database server;
b) connecting the modem and CPAP apparatus in the patient's home; and
c) causing the modem to communicate with a central database server.

20. The method according to claim 19 wherein the setting up step a) comprises the steps of by entering a modem into inventory, entering a CPAP apparatus into inventory, entering a patient, and assigning the modem and CPAP apparatus to the, patient.

21. The method according to claim 19 wherein the setting up step a) comprises the steps of by selecting a modem into inventory, selecting a CPAP apparatus into inventory, selecting a patient, and assigning the modem and CPAP apparatus to the patient.

22. The method according to claim 19 further comprising the step of entering one of either an authorized user and viewer.

23. The method according to claim 22 further comprising the step of entering one of either user and viewer rights.

24. The method according to claim 19 further comprising the step of purchasing a subscription to use the central database server.

25. The method according to claim 19 further comprising the step of accessing patient information.

26. The method according to claim 19 wherein, following step c), the method further comprising the step of notifying one of either the authorized user and viewer that the setting up step is complete.

27. The method according to claim 19 further comprising the step of the CPAP apparatus automatically communicated with the central database server.
